(19) United States
(12) Patent Application Publication
(10) Pub. No.: US 2026/0114894 A1
(43) Pub. Date: Apr. 30, 2026

(54) Tissue-Removing Catheter with Advancer Lock

(71) Applicant: Medtronic Vascular, Inc., Santa Rosa, CA (US)

(72) Inventors: Kevin Cunningham, Galway (IE); Cara MacGillivray, Gort (IE); Mark John O'Connor, Swinford (IE); Tomas K. Kelly, Galway (IE); David Betancourt, Chicago (IL); Matt Svacina, Appleton, WI (US); Brad Bogenschutz, Appleton, WI (US); Greg Aykens, Neenah, WI (US)

(21) Appl. No.: 19/120,911

(22) PCT Filed: Oct. 12, 2023
(86) PCT No.: PCT/US2023/035018

§ 371 Date: Apr. 14, 2025

Related U.S. Application Data
(60) Provisional application No. 63/379,206, filed on Oct. 12, 2022.

(51) Int. Cl. A61B 17/3207; A61B 17/00; A61B 17/32

(52) U.S. Cl. CPC A61B 17/320758; A61B 2017/00398; A61B 2017/00407; A61B 2017/320004

(57) ABSTRACT

A tissue-removing catheter includes a slide assembly which moves linearly to impart linear movement of a motor. The slide assembly includes a knob accessible outside a handle to enable the linear movement by a user. The slide lock may selectively lock the slide assembly to inhibit linear movement of the slide assembly, and selectively unlock the slide assembly to enable linear movement of the slide assembly. The knob is rotatable relative to the handle for rotating the rotatable cam such that the slide lock actuates for selectively locking and unlocking the slide assembly. The knob may rotate between first and second angular positions for unlocking and locking the slide assembly with a fixed angle of rotation to inhibit the knob from rotating beyond the second angular position in a first direction. A detent mechanism may inhibit or enable linear movement of the slide assembly.

### FIELD

The present disclosure generally relates to a tissue-removing catheter, and more particular, to a tissue-removing catheter having an advancer lock.

### BACKGROUND

Tissue-removing catheters are used to remove unwanted tissue in body lumens. As an example, atherectomy catheters are used to remove material from a blood vessel to open the blood vessel and improve blood flow through the vessel. This process can be used to prepare lesions within a patient's coronary artery to facilitate percutaneous coronary angioplasty (PTCA) or stent delivery in patients with severely calcified coronary artery lesions. Atherectomy catheters typically employ a rotating element which is used to abrade or otherwise break up the unwanted tissue.

### SUMMARY OF THE DISCLOSURE

In one aspect, a tissue-removing catheter includes an elongate body, a handle, a motor, a tissue-removing element, a slide assembly, a slide lock and a rotatable cam. The elongate body is sized and shaped to be received in the body lumen and has an axis and proximal and distal end portions spaced apart from one another along the axis. The handle is at the proximal end portion of the elongate body. The motor is in the handle and is operatively coupled to the elongate body to drive rotation of the elongate body about the axis of the elongate body. The tissue-removing element is mounted on the distal end portion of the elongate body and removes the tissue as the tissue-removing element is rotated by the elongate body within the body lumen. The slide assembly is operatively coupled to the motor and configured to selectively move linearly relative to the handle to impart linear movement of the motor within the handle. The slide assembly includes a knob accessible outside the handle and may be able to enable linear movement of the slide assembly by a user. The knob is selectively rotatable relative to the handle. The slide lock may be disposed in the handle. The slide lock may selectively lock the slide assembly to inhibit linear movement of the slide assembly relative to the handle and selectively unlock the slide assembly to enable linear movement of the slide assembly relative to the handle. The rotatable cam is operatively coupled to the knob such that rotation of the knob imparts rotation to the rotatable cam. The rotatable cam is operable to actuate the slide lock for selectively locking and unlocking the slide assembly.

In another aspect, a tissue-removing catheter for removing tissue in a body lumen generally comprises an elongate body having an axis and proximal and distal end portions spaced apart from one another along the axis. The elongate body is sized and shaped to be received in the body lumen. A handle is at the proximal end portion of the elongate body. A motor in the handle is operatively coupled to the elongate body to drive rotation of the elongate body about the axis of the elongate body. A tissue-removing element is mounted on the distal end portion of the elongate body. The tissue-removing element is configured to remove the tissue as the tissue-removing element is rotated by the elongate body within the body lumen. A slide assembly operatively is coupled to the motor and configured to selectively move linearly relative to the handle to impart linear movement of the motor within the handle. The slide assembly includes a knob accessible outside the handle and configured to enable linear movement of the slide assembly by a user. A detent mechanism is disposed in the handle. The detent mechanism is selectively operable to i) inhibit linear movement of the slide assembly relative to the handle, and ii) enable linear movement of the slide assembly relative to the handle.

In yet another aspect, a tissue-removing catheter for removing tissue in a body lumen generally comprises an elongate body having an axis and proximal and distal end portions spaced apart from one another along the axis. The elongate body is sized and shaped to be received in the body lumen. A handle is at the proximal end portion of the elongate body. A motor in the handle is operatively coupled to the elongate body to drive rotation of the elongate body about the axis of the elongate body. A tissue-removing element is mounted on the distal end portion of the elongate body. The tissue-removing element is configured to remove the tissue as the tissue-removing element is rotated by the elongate body within the body lumen. A slide assembly is operatively coupled to the motor and configured to selectively move linearly relative to the handle to impart linear movement of the motor within the handle. The slide assembly includes a knob accessible outside the handle. The knob is selectively rotatable relative to the handle in a first direction between a first angular position, in which the slide assembly is unlocked and capable of being moved linearly relative to the handle, and a second angular position, in which the slide assembly is locked and inhibited from moving linearly relative to the handle. An angle between the first angular position and the second angular position is fixed such that the knob is inhibited from rotating beyond the second angular position in the first direction.

### BRIEF DESCRIPTION OF THE DRAWINGS

FIG. 1 is a schematic illustration of a catheter of the present disclosure;

FIG. 2 is an enlarged elevation of a distal end portion of the catheter;

FIG. 3 is the enlarged elevation of a distal end portion of the catheter abrading though a lesion;

FIG. 4 is a cross section taken through line 4-4 in FIG. 2;

FIG. 5A is a top perspective of a handle of the catheter;

FIG. 5B is a top perspective of the handle with a top housing section removed;

FIG. 6 is a perspective of a gearbox housing and a motor coupled to a slide assembly and removed from the handle;

FIG. 7 is a perspective of a gear assembly in the handle;

FIG. 8A is a bottom plan view of the top housing section including a locking mechanism;

FIG. 8B is a sectional view of the top housing section including the locking mechanism;

FIG. 8C is an enlarged, partial view as indicated in FIG. 8B;

FIG. 9A is a perspective of an assembly of a slide actuator, a cam, and a detent and detent base of a detent mechanism, the detent mechanism being in an unlocked position;

FIG. 9B is similar to FIG. 9A but with the detent mechanism in a locked position;

FIG. 10 is an enlarged, fragmentary view showing teeth of the detent engaging teeth of a detent receiver;

FIG. 11 is a perspective of the detent and detent base;

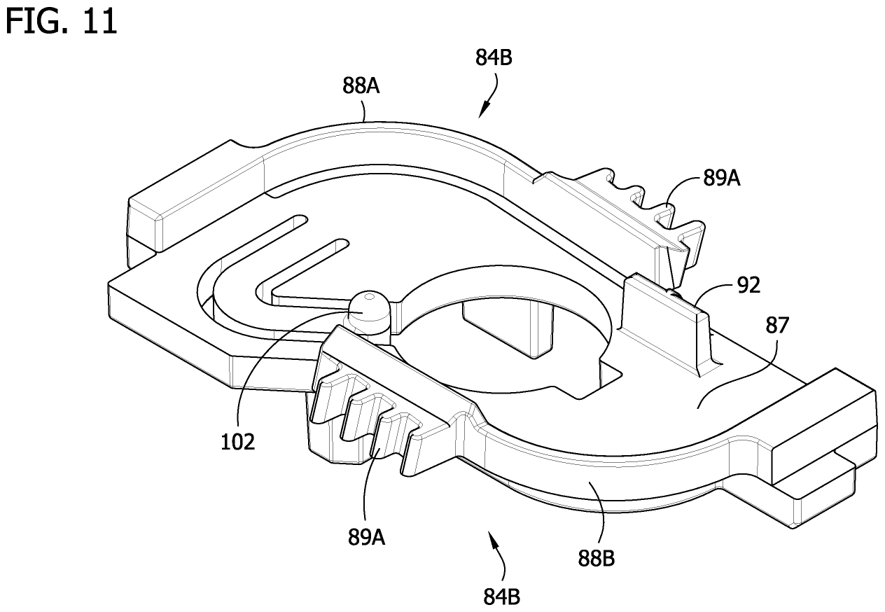

FIG. 12 is a top plan of the detent and detent base;

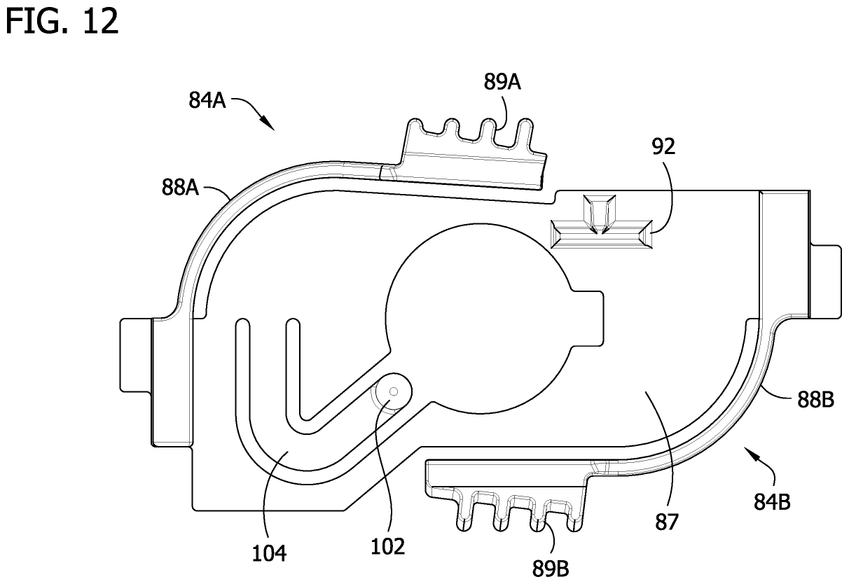

FIG. 13 is a perspective of an assembly of the cam and the slide actuator;

FIG. 14 is a bottom plan of the assembly of the cam and the slide actuator; and

FIG. 15 is an exploded view of the assembly of the slide actuator, the cam, and the detent and detent base.

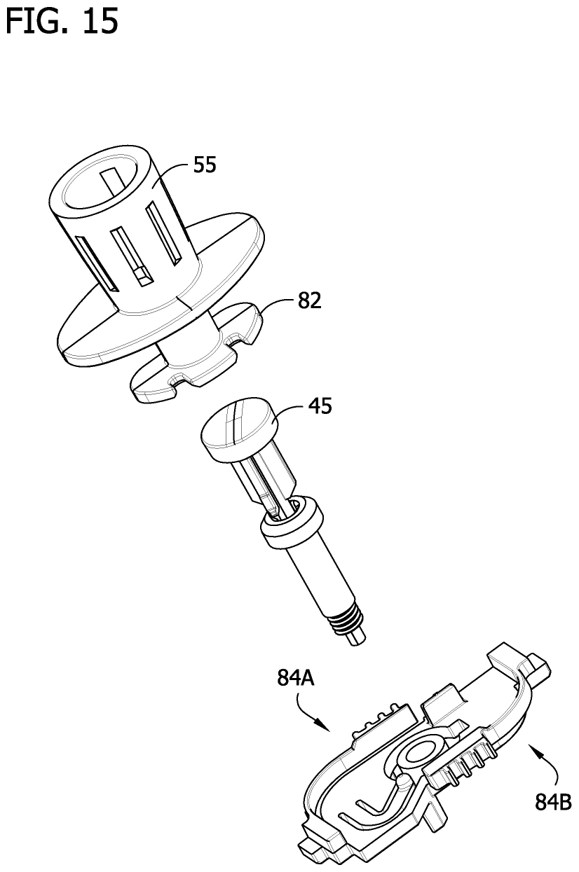

Corresponding reference characters indicate corresponding parts throughout the drawings.

### DETAILED DESCRIPTION

The present disclosure is generally directed to a tissue-removing catheter for removing tissue in a body lumen. In one embodiment, the catheter is an atherectomy device (e.g., rotational atherectomy device) suitable for removing (e.g., abrading, debulking, cutting, excising, ablating, etc.) occlusive tissue (e.g., embolic tissue, plaque tissue, atheroma, thrombolytic tissue, stenotic tissue, hyperplastic tissue, neoplastic tissue, etc.) from a vessel wall (e.g., coronary arterial wall, etc.). The catheter may be used to facilitate percutaneous coronary angioplasty (PTCA) or the subsequent delivery of a stent. Features of the disclosed embodiments may also be suitable for treating chronic total occlusion (CTO) of blood vessels and stenosis of other body lumens and other hyperplastic and neoplastic conditions in other body lumens, such as the ureter, the biliary duct, respiratory passages, the pancreatic duct, the lymphatic duct and the like. Neoplastic cell growth will often occur as a result of a tumor surrounding and intruding into a body lumen. Removal of such material can thus be beneficial to maintain patency of the body lumen.

The following is a description of a suitable tissue-removing catheter in which embodiments of the present disclosure may be incorporated. It is understood that this description of a suitable tissue-removing catheter is non-limiting, and a suitable tissue-removing catheter may omit certain component and/or include additional component(s).

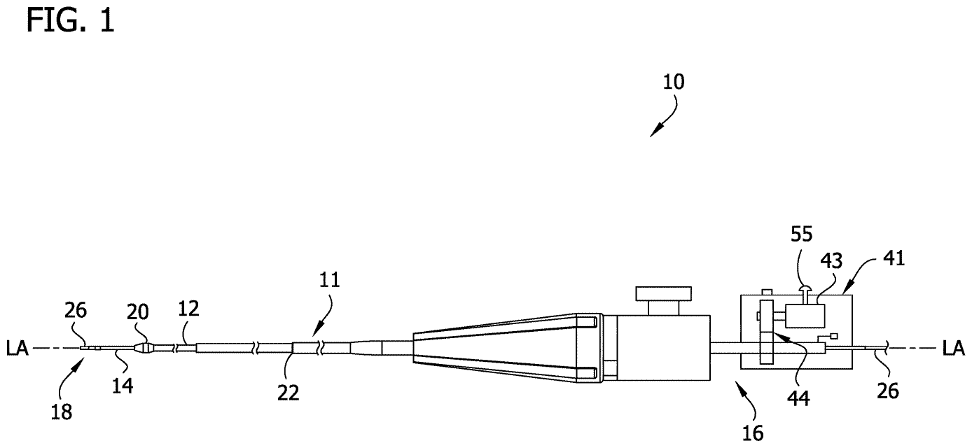

Referring to FIG. 1, an exemplary embodiment of a rotational tissue-removing catheter for removing tissue (e.g., a lesion L) in a body lumen is generally indicated at reference number 10. The illustrated catheter 10 includes an elongate catheter body, generally indicated at reference numeral 11, having proximal and distal end portions. In one example, the catheter body 11 is sized for being received in a blood vessel of a subject. Thus, the catheter body 11 may have a maximum size of 3, 4, 5, 6, 7, 8, 9, 10, or 12 French (1, 1.3, 1.7, 2, 2.3, 2.7, 3, 3.3, or 4 mm) and may have a working length of 20, 30, 40, 60, 80, 100, 120, 150, 180 or 210 cm depending of the body lumen. While the remaining discussion is directed toward a catheter for removing tissue in blood vessels, it will be appreciated that the teachings of the present disclosure also apply to other types of tissue-removing catheters, including, but not limited to, catheters for penetrating and/or removing tissue from a variety of occlusive, stenotic, or hyperplastic material in a variety of body lumens.

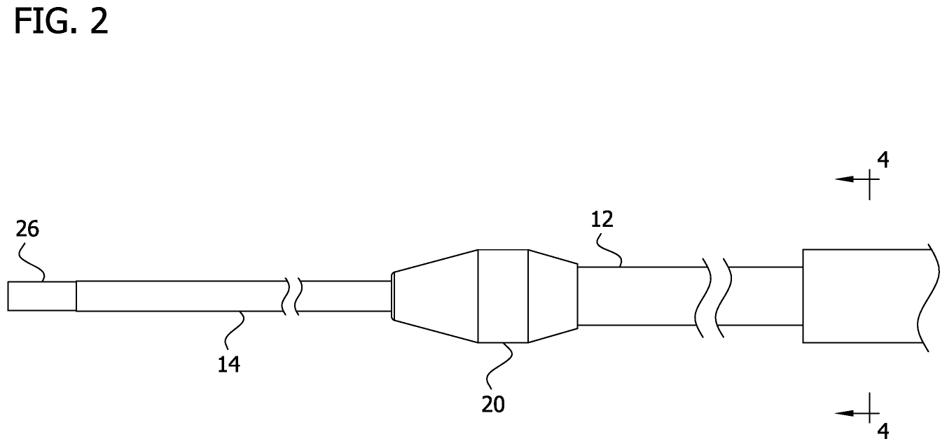

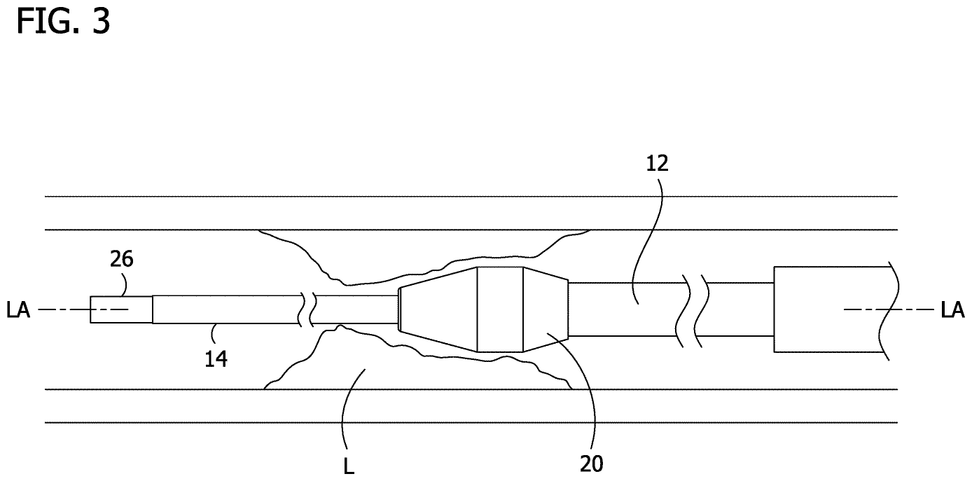

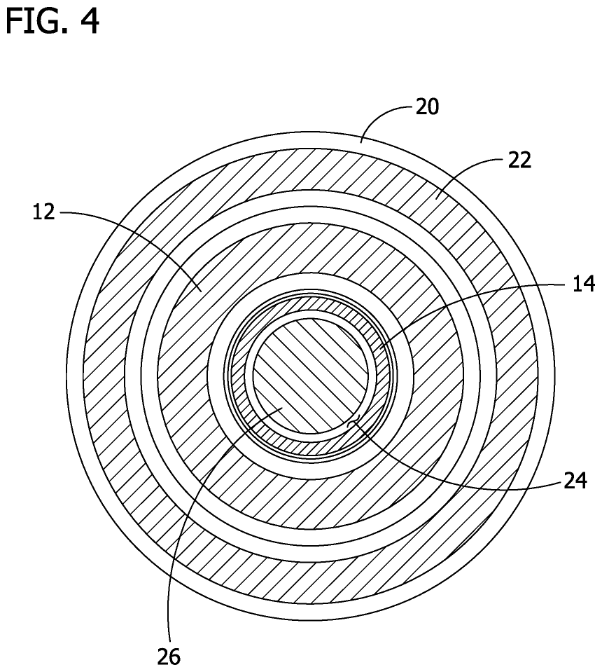

Referring to FIGS. 1-4, the catheter body 11 comprises an elongate drive shaft or drive coil 12 (broadly, an elongate body) disposed around an elongate inner liner 14. The drive coil 12 and inner liner 14 extend along a longitudinal axis LA of the catheter from a proximal end portion 16 to a distal end portion 18 of the catheter. A tissue-removing element 20 (e.g. an abrasive burr) is disposed on a distal end portion of the catheter body 11 (e.g., on the drive coil 12). The tissue-removing element 20 is configured for rotation by rotation of the drive coil 12 to remove tissue, such as a lesion L, from a body lumen. The abrasive burr 20 may have an abrasive outer surface formed, for example, by a diamond grit coating, surface etching, or the like. A sheath 22 (FIG. 1) is disposed around the drive coil 12. The drive coil 12 and the inner liner 14 are both configured to translate relative to the sheath 22 using a slide assembly, as described below. The catheter 10 is sized and shaped for insertion into a body lumen of a subject. The sheath 22 isolates the body lumen from at least a portion of the drive coil 12 and inner liner 14. The inner liner 14 defines a guidewire lumen 24 for slidably receiving a guidewire 26 therein so that the catheter 10 can be advanced through the body lumen by traveling along the guidewire. The guidewire can be a standard 0.014-inch outer diameter, 300 cm length guidewire. In certain embodiments, the inner liner 14 may have a lubricious inner surface for sliding over the guidewire 26 (e.g., a lubricious surface may be provided by a lubricious polymer layer or a lubricious coating). In the illustrated embodiment, the guidewire lumen 24 (FIG. 4) extends from the proximal end portion 16 through the distal end portion 18 of the catheter 10 such that the guidewire 26 is extendable along an entire working length of the catheter 10. In one embodiment, the overall working length of the catheter 10 may be between about 130 cm (51.2 inches) and about 160 cm (63 inches). In use, the guidewire 26 may extend about 40 mm (1.6 inches) past a distal end of the inner liner 14.

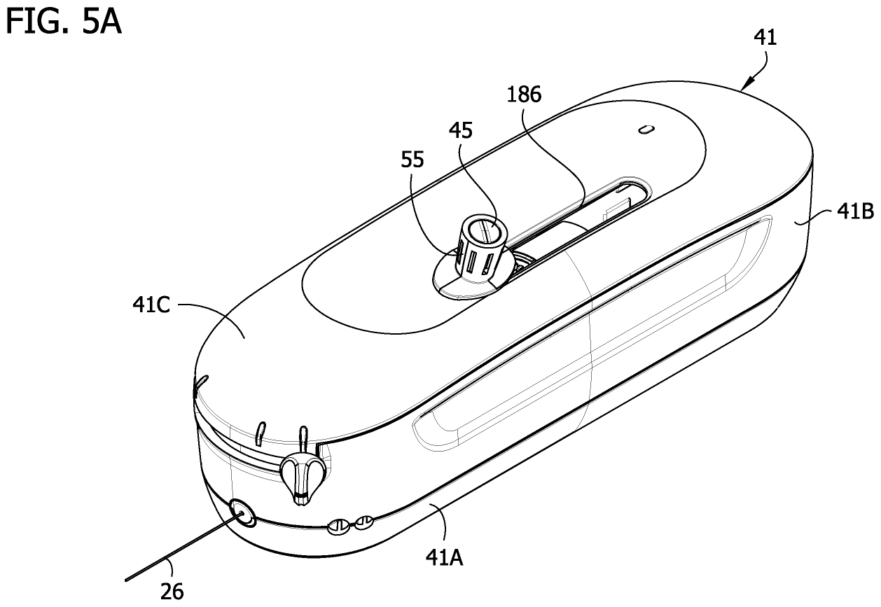

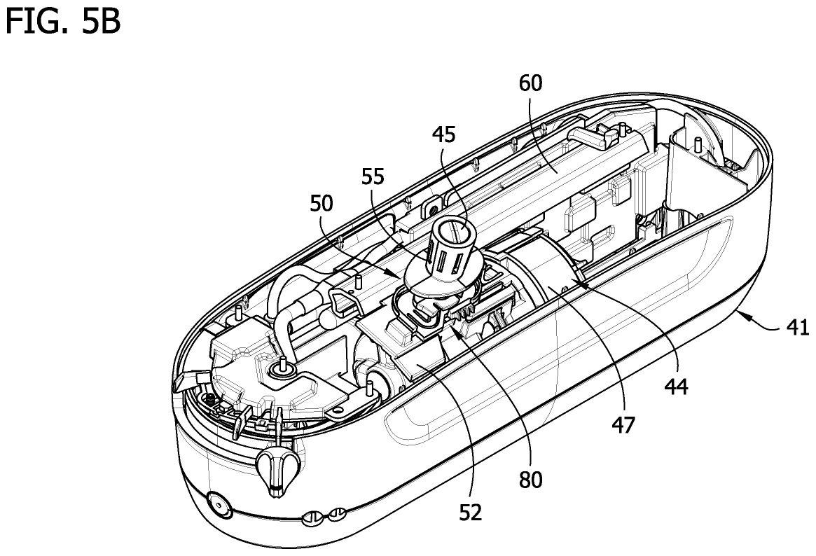

Referring to FIGS. 5A and 5B, the catheter 10 further comprises a handle, generally indicated at 41, coupled to a proximal end of the catheter body 11, such as at the isolation sheath 22. The handle 41 is configured to support components therein. In the illustrated embodiment, the handle 41 includes a bottom housing section 41A, a middle housing section 41B secured to the top of the bottom housing section, and a top housing section 41C secured to the top of the middle housing section. It will be understood that the handle 41 can have other shapes and configurations without departing from the scope of the disclosure.

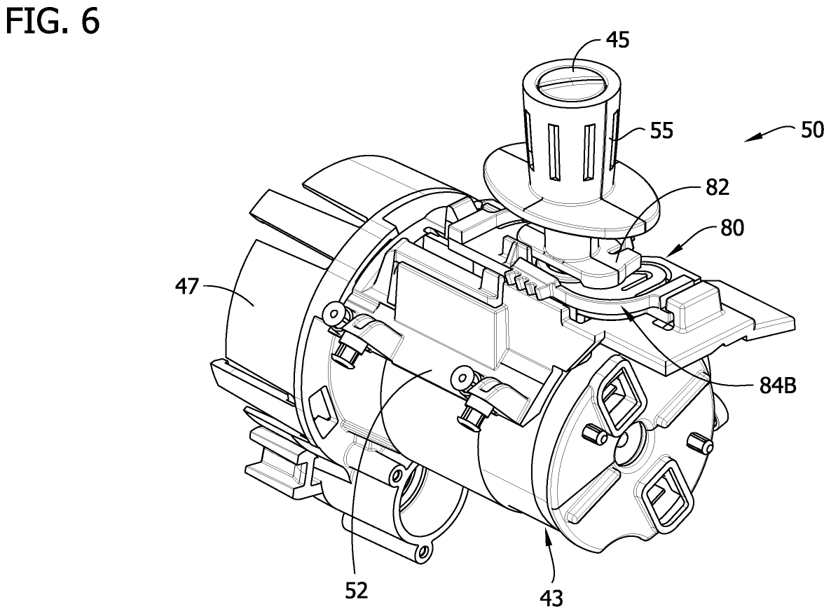

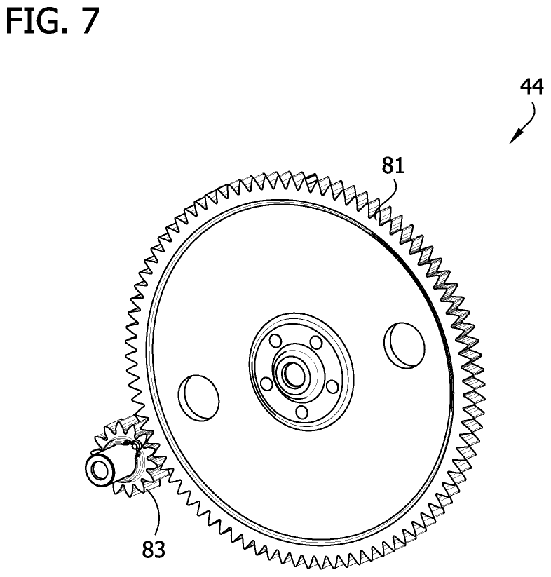

Referring to FIGS. 5A and 5B, the handle 41 supports a motor actuator 45 (e.g., a lever, a button, a dial, a switch, or other device) configured for selectively actuating a motor 43 (e.g., an electric motor), disposed in the handle to drive rotation of the elongate body (e.g., the drive coil 12) and the tissue-removing element 20 mounted at the distal end of the elongate body. The motor 43 is configured to rotate the drive coil 12 and the tissue-removing element 20 at speeds of up to, for example but not limiting to, about 100,000 RPM. In the illustrated embodiment, the motor 43 is coupled to the drive coil 12 by a gear assembly 44 and drive assembly (broadly, a motor drivetrain output) supported within the handle 41. Referring to FIGS. 6 and 7, the gear assembly 44 comprises a gearbox housing 47 (FIG. 6) that mounts and at least partially encloses a pair of gears 81, 83 (FIG. 7) for transferring the rotation of a shaft of the motor 43 to the drive coil 12. In particular, a driven gear 83 is in mesh with the driver gear 81 so that rotation of the driver gear causes the driven gear to rotate in the opposite direction. Thus, when current is applied to the motor 43 from a power source, the electrical energy from the current is converted into mechanical energy to rotate the gear assembly 44 for rotating the drive coil 12. It is understood that the motor 43 may be coupled to the elongate body (e.g., drive coil 12) in other ways for transferring rotation (torque) from the motor to the elongate body.

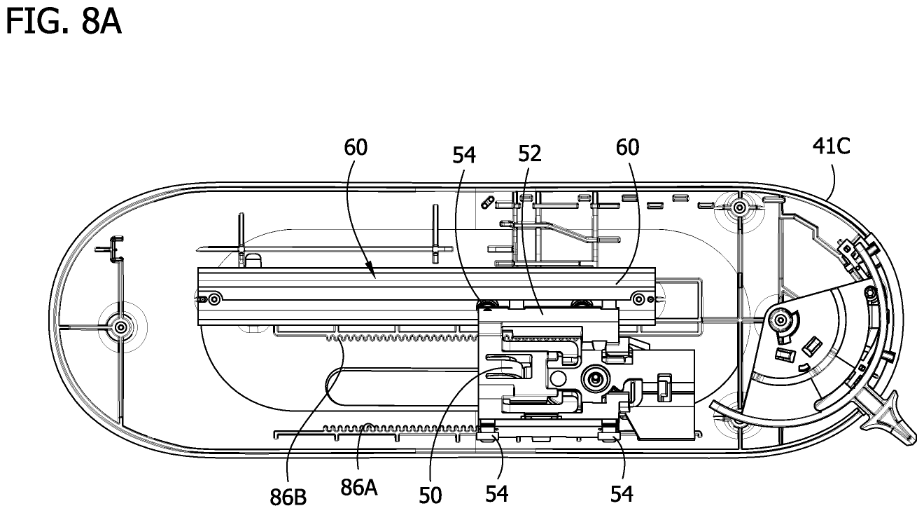

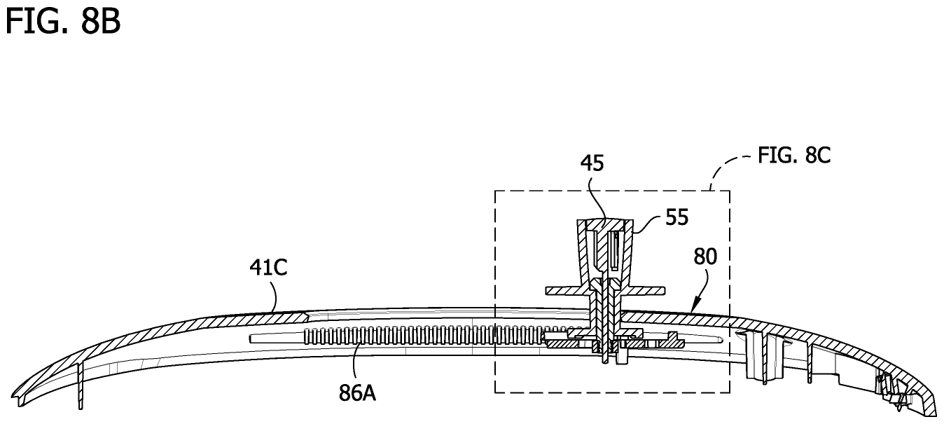

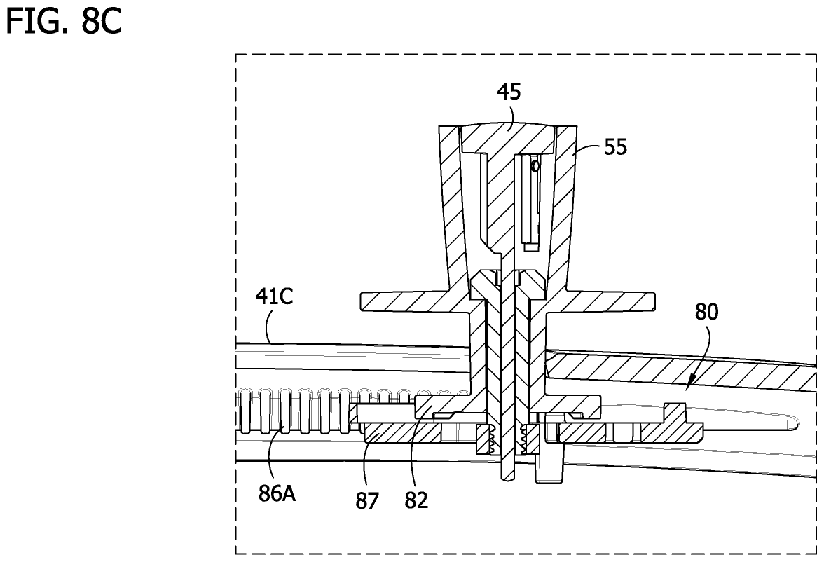

Referring to FIGS. 6 and 8A, the tissue-removing catheter 10 includes a slide assembly, generally indicated at 50, in the handle 41. The slide assembly 50 includes a carriage 52 coupled to and supporting at least the motor 43, and a slide actuator 55 coupled to the carriage to enable a user to operate the slide assembly. The slide assembly 50 enables a user to linearly advance the motor 43 (and the gear assembly 44 in one embodiment) within the handle 41 to impart linear movement to the drive coil 12 and the tissue-removing element 20 relative to the handle. For example, the slide assembly 50 may allow from about 30 mm to about 70 mm or more of linear advancement of the tissue-removing element 20. In the illustrated embodiment, the slide assembly 50 further includes a track (e.g., rail 60; FIG. 5B) to which the carriage 52 is coupled to enable linear movement of the carriage relative to the handle. As an example, bearings 54 (e.g., roller bearings) may couple the carriage to the track. In the illustrate embodiment, the slide actuator 55 comprises a knob to enable a user's hand to manually operate the slide assembly 50. The motor actuator 42 is coupled to the slide actuator 55 in the illustrated embodiment. In one or more other embodiments, the motor actuator may be located elsewhere on the handle or separate from the handle.

Referring to FIGS. 6, 8A-8C, the catheter 10 further includes a slide lock, generally indicated at 80, disposed in the handle 41. The slide lock 80 is configured to selectively lock the slide assembly 50 to inhibit linear movement of the slide assembly relative to the handle 41, and selectively unlock the slide assembly to enable linear movement of the slide assembly relative to the handle. As explained in more detail below, the illustrated slide actuator 55 is rotatable relative to the handle 41 (and the carriage 52) to actuate operation of the slide lock 80. In particular, the illustrated catheter 10 includes a rotatable cam 82 operatively coupled to the actuator 55 such that rotation of the actuator (e.g., knob) relative to the handle 41 imparts rotation to the rotatable cam 82. In turn, the rotatable cam 82 is operable to actuate the slide lock 80 for selectively locking and unlocking the slide assembly 50. The rotatable cam 82 is coupled to the actuator 55 (and the carriage 52) of the slide assembly 50 such that the rotatable cam is linearly moveable with the actuator and the carriage. The slide lock 80 and the rotatable cam 82 together form a locking mechanism to allow a user to selectively lock the slide assembly 50 relative to the handle 41.

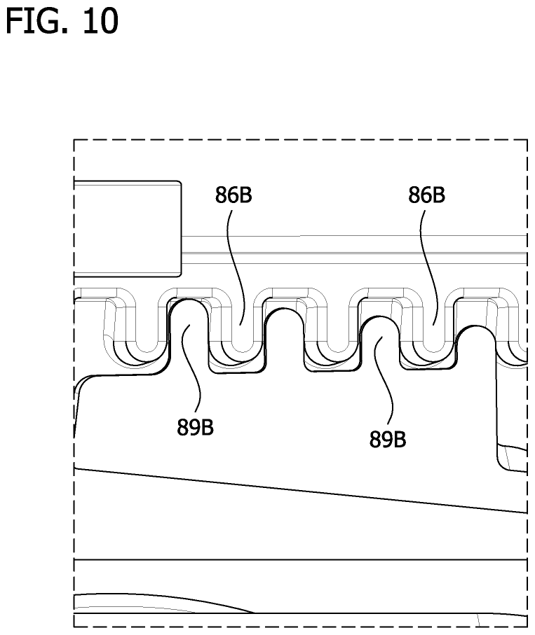

The illustrated slide lock 80 comprises a detent mechanism, as shown in FIGS. 8B and 8C. The detent mechanism includes at least one detent (e.g., first and second detents generally indicated at 84A, 84B, respectively) and at least one detent receiver (e.g., first and second detent receivers generally indicated at 86A, 86B, respectively) configured to selectively receive the at least one detent to lock the slide assembly 50. The illustrated detents 84A, 84B are coupled to (e.g., integrally formed with) a detent base 87 of the detent mechanism, which is in turn coupled to the advancer 55 such that the detents 84A, 84B linearly move with the actuator and the carriage 52. The illustrated detents 84A, 84B are pawls, each including a pawl arm 88A, 88B (broadly, a locking arm) coupled to the base 87, and a row of teeth 89A, 89B on the corresponding pawl arm. As explained below, the pawl arms 88A, 88B are resiliently deflectable in a lateral direction relative to the base 87. As shown in FIG. 8A, the illustrated detent receivers 86A, 86B are linear racks, each of which is coupled to the handle 41. The linear racks 86A, 86B extend along the inside of the handle 41 and may be formed integrally therewith. The linear racks 86A, 86B each include a row of teeth configured to engage (i.e., mesh with) the corresponding pawl teeth 89A, 89B to selectively lock the slide assembly 50, as shown in FIG. 10. The detents 84A, 84B and detent receivers 86A, 86B may be of other designs and constructions.

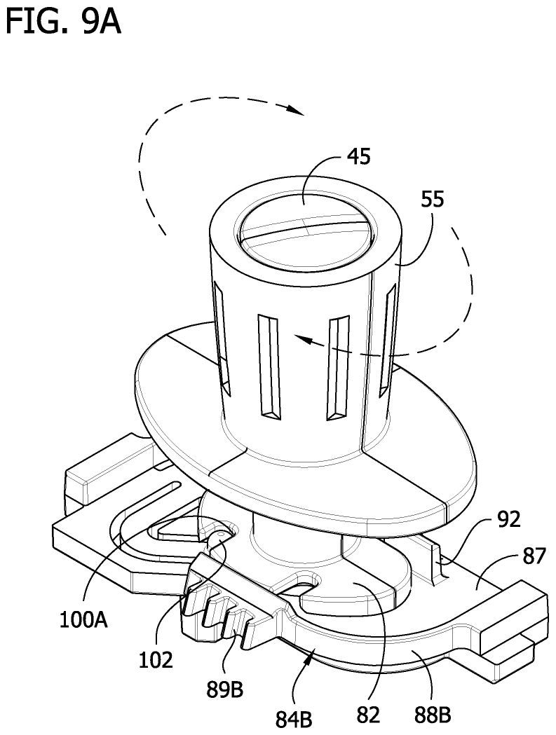

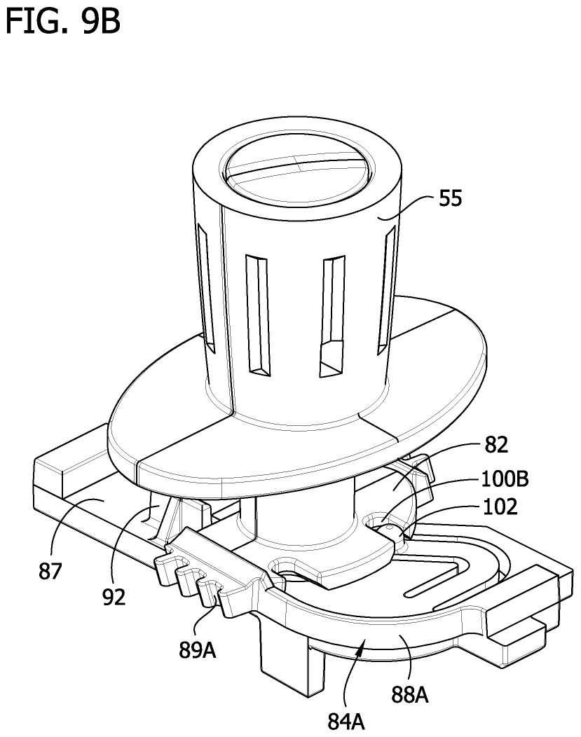

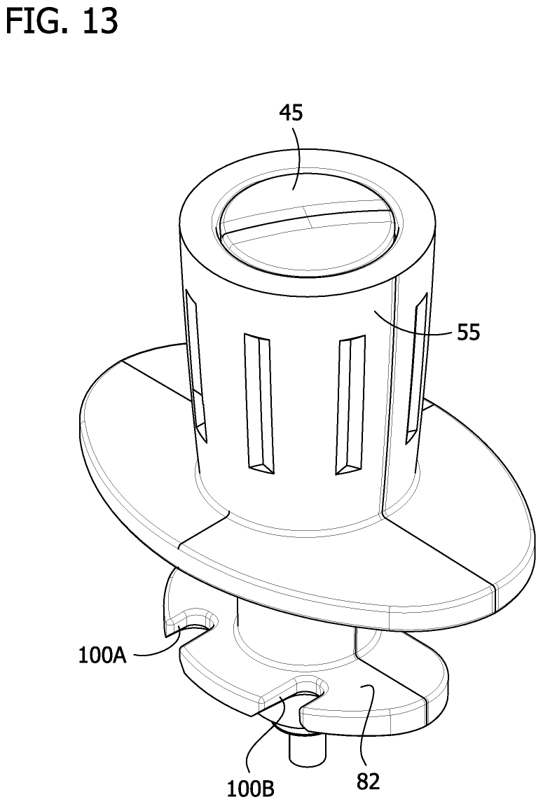

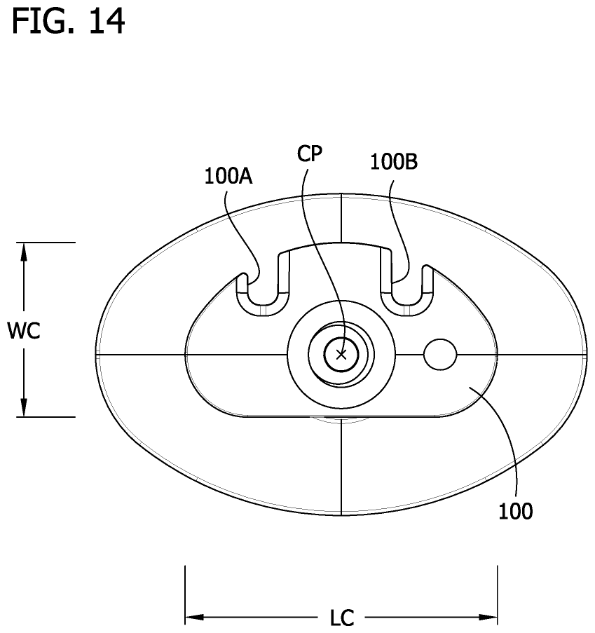

Referring to FIGS. 13 and 14, the illustrated cam 82 has a non-symmetrical, elongate footprint. In particular, as shown in FIG. 14 the illustrated cam 82 is a disc having opposite rounded longitudinal ends, an arcuate first side, and a generally flat second side. The cam 82 has a length LC between the longitudinal ends, and a width WC between the first and second sides. As shown in FIG. 9A, in a first angular position the arcuate and flat sides of the cam 82 generally oppose adjacent ones of the detents 84A, 84B (e.g., first and second pawl teeth 89A, 89B). The width WC of the cam 82 is such that the detents 84A, 84B are not received in the detent receivers 86A, 86B (e.g., the pawls are disengaged from the racks) when the cam is in the first angular position. Accordingly, in this first angular position of the cam 82, the slide assembly 50 is unlocked and capable of translating relative to the handle 41. As shown in FIG. 9B, when the cam 82 is rotated to a second angular position, such as by rotating the actuator 55, the longitudinal ends of the cam engage and push the corresponding detents 84A, 84B (e.g., pawls) lateral outward to locked positions so that the detents are received in the corresponding detent receivers (e.g., the pawls are engaged with the racks). Accordingly, in this second angular position of the cam 82, the slide assembly 50 is locked and inhibited from translating relative to the handle 41. It is believed this engagement or meshing inhibits incidental slipping or movement of the slide assembly as compared to a lock that relies solely upon frictional engagement or clamping on the housing. When the cam 82 is rotated back to the first angular position from the second angular position, the detents (e.g., pawls) 84A, 84B rebound to their initial, unlocked positions. In particular, the illustrated pawl arms 88A, 88B are resilient and “spring back”to the unlocked positions when the pushing force from the cam 82 is removed. Through this design, the user is aware of the slide assembly 50 being unlocked when the actuator 55 is rotated to this position by the fact that the actuator is in this rotational position. This is not necessarily possible with a design where a lock that relies solely upon frictional engagement or clamping on the housing.

As explained above, rotation of the actuator 55 imparts rotation to the cam 82 between the first and second angular positions. The actuator 55 and the cam 82 are rotatable in a first direction from the first angular position to the second angular position to lock the slide assembly. The actuator 55 and the cam 82 are rotatable in a second direction, opposite the first direction, from the second angular position to the first angular position to unlock the slide assembly 50. In the illustrated embodiment, the included angle between the first and second angular positions is fixed such that the actuator 55 and cam 82 are inhibited from rotating beyond the second angular position in the first direction and the actuator 55 and cam 82 are inhibited from rotating beyond the first angular position in the second direction. Through this configuration, the user is aware of when the slide assembly 50 is locked and unlocked by observing the rotational position of the actuator 55. Moreover, use merely needs to rotate the actuator less than 180 degrees (e.g., 90 degrees) to lock and unlock the slide assembly 50, rather than continuing to rotate the actuator more than 180 degrees or some other non-definite amount to clamp the actuator on the handle. The slide lock 80 includes a stop 92 (e.g., a post) on the base 87. The cam 82 (e.g., the flat side of the cam) engages the stop 92 when the cam is in the first angular position and the second angular position, thereby inhibiting rotation outside the included angle between the first and second angular positions. In the illustrated embodiment, the included angle is less than 360 degrees, such as less than 180 degrees, for example about 90 degrees.

The illustrated locking mechanism (i.e., the slide lock 80 in combination with the cam) is configured to releasably lock the cam 82 in the first and second angular positions. The locking mechanism may also be configured provide feedback (tactile and/or auditory feedback) to the user upon the slide lock 80 moving to the locked and/or unlocked positions. The cam 82 defines at least one notch (e.g., first and second notches 100A, 100B) extending inward from the round side of the cam. As shown in FIG. 9A, the notch 100A is configured to receive a pin 102 by snap-fit connection when the cam is rotated in the second direction to the first angular position. The pin 102 snaps into the notch 100A upon the cam 82 being in the first angular position, thereby releasably locking the cam in the first angular position and indicating to the user by tactile and/or auditory feedback that the slide assembly is unlocked. The pin 102 is configured to snap out of the notch 100A (i.e., unsnap) upon the cam being rotated by a suitable torque in the first direction and away from the first angular position toward the second angular position. A shown in FIG. 9B, the notch 100B is configured to receive the pin 102 by snap-fit connection when the cam is rotated in the first direction to the second angular position. The pin 102 snaps into the notch 100B upon the cam 82 being in the second angular position, thereby releasably locking the cam in the second angular position and indicating to the user by tactile and/or auditory feedback that the slide assembly is locked. The pin 102 is configured to snap out of the notch 100B (i.e., unsnap) upon the cam being rotated by a suitable amount of torque in the second away from the second angular position toward the first angular position. This tactile and/or auditory feedback when locking and/or unlocking the slide assembly 50 provides an indication to the user as to the locked state of the slide assembly being locked and/or unlocked, contrary to a lock that does not include any feedback to indicate the locked state of the slide assembly. In the illustrated embodiment, the pin 102 is resiliently deflectable (e.g., depressible) to allow the cam 82 to slide over the pin as the cam is rotated between the first and second angular positions.

In use, the slide lock 80 is operable to selectively lock and unlock the slide assembly 50. In one exemplary method of use, the tissue-removing element 20 is delivered to the treatment site and is rotated by the motor 43 to remove unwanted tissue from the body lumen. The user may selectively operate the slide assembly 50 by moving the slide actuator 55 relative to the handle 41 to translate the tissue-removing element 20 relative to the handle 41. The use may operate the slide lock 80 to temporarily fix the longitudinal position of the tissue-removing element 20 relative to the handle 41. The user may further unlock the slide lock 80 to enable translation of the tissue-removing element 20 relative to the handle. When unlocked, the user may move the slide actuator 55 forward and then backward to create a “pecking” motion at the tissue-removing element 20. The tissue-removing catheter 10 may be used in other ways suitable for removing tissue from a body lumen.

The invention may be further described by reference to the following numbered paragraphs:
1. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen;
   a slide assembly operatively coupled to the motor and configured to selectively move linearly relative to the handle to impart linear movement of the motor within the handle, the slide assembly including a knob accessible outside the handle and configured to enable linear movement of the slide assembly by a user, wherein the knob is selectively rotatable relative to the handle;
   a slide lock disposed in the handle, the slide lock configured to i) selectively lock the slide assembly to inhibit linear movement of the slide assembly relative to the handle, and ii) selectively unlock the slide assembly to enable linear movement of the slide assembly relative to the handle; and
   a rotatable cam operatively coupled to the knob such that rotation of the knob imparts rotation to the rotatable cam, wherein the rotatable cam is operable to actuate the slide lock for selectively locking and unlocking the slide assembly.
2. The tissue-removing catheter set forth in paragraph 1, wherein the slide lock and the rotatable cam are coupled to the slide assembly such that the slide lock and the rotatable cam are linearly movable with the slide assembly.
3. The tissue-removing catheter set forth in paragraph 1, wherein the slide lock includes at least one locking arm configured to be moved to a locking position by the rotatable cam to lock the slide assembly.
4. The tissue-removing catheter set forth in paragraph 3, wherein the at least one locking arm comprises opposing first and second locking arms.
5. The tissue-removing catheter set forth in paragraph 4, wherein the first and second locking arms are simultaneously displaceable by the rotatable cam.
6. The tissue-removing catheter set forth in paragraph 3, wherein the at least one locking arm is resiliently biased in an unlocked position in which the slide assembly is unlocked.
7. The tissue-removing catheter set forth in paragraph 3, further comprising a detent receiver in the housing, wherein the at least one locking arm comprises a detent configured to be received in the detent receiver to lock the slide assembly.
8. The tissue-removing catheter set forth in paragraph 1, wherein the slide lock comprises a detent mechanism.
9. The tissue-removing catheter set forth in paragraph 6, wherein the knob is selectively rotatable relative to the handle in a first direction between a first angular position, in which the cam is rotated to a first angular position and the locking arm is in the unlocked position, and a second angular position, in which the cam is rotated to a second angular position and the locking arm is in the locked position.
10. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen;
   a slide assembly operatively coupled to the motor and configured to selectively move linearly relative to the handle to impart linear movement of the motor within the handle, the slide assembly including a knob accessible outside the handle and configured to enable linear movement of the slide assembly by a user; and
   a detent mechanism disposed in the handle, the detent mechanism being selectively operable to i) inhibit linear movement of the slide assembly relative to the handle, and ii) enable linear movement of the slide assembly relative to the handle.
11. The tissue-removing catheter set forth in paragraph 10, wherein the detent mechanism includes a detent coupled to and movable with the slide assembly, and a detent receiver coupled to the handle, wherein the detent is configured to be removably received in the detent receiver.
12. The tissue-removing catheter set forth in paragraph 11, wherein the detent includes at least one pawl having at least one tooth, wherein the detent receiver includes at least one linear rack having a plurality of teeth, wherein the at least one pawl is selectively movable between a locked position, in which the pawl is engaged with the linear rack, and an unlocked position, in which the pawl is disengaged from the linear rack.
13. The tissue-removing catheter set forth in paragraph 12, wherein the at least one pawl comprises first and second pawls, wherein the at least one linear rack comprises first and second racks, wherein the first pawl is selectively engageable with the first linear rack and the second pawl is selectively engageable with the second linear rack.
14. The tissue-removing catheter set forth in paragraph 12, wherein the pawl is resiliently deflectable from the unlocked position to the locked position.
15. The tissue-removing catheter set forth in paragraph 14, further comprising a rotatable cam operable to resiliently deflect the pawl from the unlocked position to the locked position, wherein the knob is rotatable relative to the handle and is operatively coupled to the cam such that rotation of the knob imparts rotation to the rotatable cam to resiliently deflect the pawl from the unlocked position to the locked position.
16. The tissue-removing catheter set forth in paragraph 15, wherein the knob is selectively rotatable relative to the handle in a first direction between a first angular position, in which the cam is rotated to a first angular position and the pawl is in the unlocked position, and a second angular position, in which the cam is rotated to a second angular position and the pawl is in the locked position.
17. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen;
   a slide assembly operatively coupled to the motor and configured to selectively move linearly relative to the handle to impart linear movement of the motor within the handle, the slide assembly including a knob accessible outside the handle,
   wherein the knob is selectively rotatable relative to the handle in a first direction between a first angular position, in which the slide assembly is unlocked and capable of being moved linearly relative to the handle, and a second angular position, in which the slide assembly is locked and inhibited from moving linearly relative to the handle,
   wherein an angle between the first angular position and the second angular position is fixed such that the knob is inhibited from rotating beyond the second angular position in the first direction.
18. The tissue-removing catheter set forth in paragraph 17, wherein the angle between the first angular position and the second angular position is about 90 degrees.
19. The tissue-removing catheter set forth in paragraph 17, wherein the knob is configured to be releasably locked in the first and second angular positions.
20. The tissue-removing catheter set forth in paragraph 19, further comprising a snap-fit connection configured to releasably lock the knob in the first and second angular positions.

When introducing elements of the present invention or the one or more embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

1-20. (canceled)

21. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen;
a slide assembly operatively coupled to the motor and configured to selectively move linearly relative to the handle to impart linear movement of the motor within the handle, the slide assembly including a knob accessible outside the handle and configured to enable linear movement of the slide assembly by a user, wherein the knob is selectively rotatable relative to the handle;
a slide lock disposed in the handle, the slide lock configured to i) selectively lock the slide assembly to inhibit linear movement of the slide assembly relative to the handle, and ii) selectively unlock the slide assembly to enable linear movement of the slide assembly relative to the handle; and
a rotatable cam operatively coupled to the knob such that rotation of the knob imparts rotation to the rotatable cam, wherein the rotatable cam is operable to actuate the slide lock for selectively locking and unlocking the slide assembly.

22. The tissue-removing catheter set forth in claim 21, wherein the slide lock and the rotatable cam are coupled to the slide assembly such that the slide lock and the rotatable cam are linearly movable with the slide assembly.

23. The tissue-removing catheter set forth in claim 21, wherein the slide lock includes at least one locking arm configured to be moved to a locking position by the rotatable cam to lock the slide assembly.

24. The tissue-removing catheter set forth in claim 23, wherein the at least one locking arm comprises opposing first and second locking arms.

25. The tissue-removing catheter set forth in claim 24, wherein the first and second locking arms are simultaneously displaceable by the rotatable cam.

26. The tissue-removing catheter set forth in claim 23, wherein the at least one locking arm is resiliently biased in an unlocked position in which the slide assembly is unlocked.

27. The tissue-removing catheter set forth in claim 23, further comprising a detent receiver in the housing, wherein the at least one locking arm comprises a detent configured to be received in the detent receiver to lock the slide assembly.

28. The tissue-removing catheter set forth in claim 21, wherein the slide lock comprises a detent mechanism.

29. The tissue-removing catheter set forth in claim 26, wherein the knob is selectively rotatable relative to the handle in a first direction between a first angular position, in which the cam is rotated to a first angular position and the locking arm is in the unlocked position, and a second angular position, in which the cam is rotated to a second angular position and the locking arm is in the locked position.

30. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen; and
a slide assembly operatively coupled to the motor and configured to selectively move linearly relative to the handle to impart linear movement of the motor within the handle, the slide assembly including a knob accessible outside the handle and configured to enable linear movement of the slide assembly by a user; and
a detent mechanism disposed in the handle, the detent mechanism being selectively operable to i) inhibit linear movement of the slide assembly relative to the handle, and ii) enable linear movement of the slide assembly relative to the handle.

31. The tissue-removing catheter set forth in claim 30, wherein the detent mechanism includes a detent coupled to and movable with the slide assembly, and a detent receiver coupled to the handle, wherein the detent is configured to be removably received in the detent receiver.

32. The tissue-removing catheter set forth in claim 31, wherein the detent includes at least one pawl having at least one tooth, wherein the detent receiver includes at least one linear rack having a plurality of teeth, wherein the at least one pawl is selectively movable between a locked position, in which the pawl is engaged with the linear rack, and an unlocked position, in which the pawl is disengaged from the linear rack.

33. The tissue-removing catheter set forth in claim 32, wherein the at least one pawl comprises first and second pawls, wherein the at least one linear rack comprises first and second racks, wherein the first pawl is selectively engageable with the first linear rack and the second pawl is selectively engageable with the second linear rack.

34. The tissue-removing catheter set forth in claim 32, wherein the pawl is resiliently deflectable from the unlocked position to the locked position.

35. The tissue-removing catheter set forth in claim 34, further comprising a rotatable cam operable to resiliently deflect the pawl from the unlocked position to the locked position, wherein the knob is rotatable relative to the handle and is operatively coupled to the cam such that rotation of the knob imparts rotation to the rotatable cam to resiliently deflect the pawl from the unlocked position to the locked position.

36. The tissue-removing catheter set forth in claim 35, wherein the knob is selectively rotatable relative to the handle in a first direction between a first angular position, in which the cam is rotated to a first angular position and the pawl is in the unlocked position, and a second angular position, in which the cam is rotated to a second angular position and the pawl is in the locked position.

37. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen;
a slide assembly operatively coupled to the motor and configured to selectively move linearly relative to the handle to impart linear movement of the motor within the handle, the slide assembly including a knob accessible outside the handle,
wherein the knob is selectively rotatable relative to the handle in a first direction between a first angular position, in which the slide assembly is unlocked and capable of being moved linearly relative to the handle, and a second angular position, in which the slide assembly is locked and inhibited from moving linearly relative to the handle,
wherein an angle between the first angular position and the second angular position is fixed such that the knob is inhibited from rotating beyond the second angular position in the first direction.

38. The tissue-removing catheter set forth in claim 37, wherein the angle between the first angular position and the second angular position is about 90 degrees.

39. The tissue-removing catheter set forth in claim 37, wherein the knob is configured to be releasably locked in the first and second angular positions.

40. The tissue-removing catheter set forth in claim 39, further comprising a snap-fit connection configured to releasably lock the knob in the first and second angular positions.
